(19) United States
(12) Patent Application Publication
(10) Pub. No.: US 2016/0024573 A1
(43) Pub. Date: Jan. 28, 2016

(54) PHOTOCLEAVABLE DEOXYNUCLEOTIDES WITH HIGH-RESOLUTION CONTROL OF DEPROTECTION KINETICS

(71) Applicant: IBIS BIOSCIENCES, INC., Carlsbad, CA (US)

(72) Inventors: Jeffrey Huff, Carlsbad, CA (US); Mark A. Hayden, Carlsbad, CA (US)

(21) Appl. No.: 14/775,072

(22) PCT Filed: Mar. 12, 2014
(86) PCT No.: PCT/US14/24379

§ 371 Date: Sep. 11, 2015

Related U.S. Application Data
(60) Provisional application No. 61/791,774, filed on Mar. 15, 2013.

(51) Int. Cl. C12Q 1/68; C07H 19/10

(52) U.S. Cl. CPC C12Q 1/6874; C07H 19/10; C12Q 1/6806

(57) ABSTRACT

Provided herein are new classes of photocleavable deoxynucleotides that allow for more precise control over deprotection kinetics compared to previously described compounds. The compounds further feature more favorable solubility properties. The nucleotides find use in methods such as next-generation sequencing. A series of molecules are provided with defined organic substituents that allow fine tuning of the deprotection kinetics when irradiated with an appropriate light source.

This application claims priority to U.S. provisional patent application Ser. No. 61/791,774, filed Mar. 15, 2013, which is incorporated herein by reference in its entirety.

### FIELD

Provided herein are new classes of photocleavable deoxynucleotides that allow for more precise control over deprotection kinetics compared to previously described compounds. The compounds further feature more favorable solubility properties. The nucleotides find use in methods such as next-generation sequencing. A series of molecules are provided with defined organic substituents that allow fine tuning of the deprotection kinetics when irradiated with an appropriate light source.

### BACKGROUND

DNA sequencing is driving genomics research and discovery. The completion of the Human Genome Project was a monumental achievement with incredible amount of combined efforts among genome centers and scientists worldwide. This decade-long project was completed using the Sanger sequencing method, which remains the staple genome sequencing methodology in high-throughput genome sequencing centers. The main reason behind the prolonged success of this method is its basic and efficient, yet elegant, method of dideoxy chain termination. With incremental improvements in Sanger sequencing—including the use of laser-induced fluorescent excitation of energy transfer dyes, engineered DNA polymerases, capillary electrophoresis, sample preparation, informatics, and sequence analysis software—the Sanger sequencing platform has been able to maintain its status. Current state-of-the-art Sanger based DNA sequencers can produce over 700 bases of clearly readable sequence in a single run from templates up to 30 kb in length. However, as it is with most technological inventions, the continual improvements in this sequencing platform has come to a stagnant plateau, with the current cost estimate for producing a high-quality microbial genome draft sequence at around $10,000 per megabase pair. Current DNA sequencers based on the Sanger method allow up to 384 samples to be analyzed in parallel.

It is evident that exploiting the complete human genome sequence for clinical medicine and health care requires accurate low-cost and high-throughput DNA sequencing methods. Indeed, both public (National Human Genome Research Institute, NHGRI) and private genomic sciences sector (The J. Craig Venter Science Foundation and Archon X prize for genomics) have issued a call for the development of “next-generation” sequencing technology that will reduce the cost of sequencing to one-ten thousandth of its current cost over the next ten years. Accordingly, to overcome the limitations of current conventional sequencing technologies, a variety of new DNA sequencing methods have been investigated, including sequencing-by-synthesis (SBS) approaches such as pyrosequencing (Ronaghi et al. (1998) Science 281: 363-365), sequencing of single DNA molecules (Braslaysky et al. (2003) Proc. Natl. Acad. Sci. USA 100: 3960-3964), and polymerase colonies (“polony” sequencing) (Mitra et al. (2003) Anal. Biochem. 320: 55-65).

Some conventional next-generation sequencing technologies include single molecule optical detection methods, e.g., as used in technologies developed by PacBio; optical (clonal) methods, e.g., as used in technologies developed by Illumina; and fluorescently labeled nucleotide based methods (including those that use photodeprotection), e.g., as used in technology developed by Lasergen. Such methods have varying degrees of advantages and disadvantages, but the significant challenge up until now has remained the issue of conducting such sequencing analyses with ultra-low cost instrumentation systems with truly low cost and disposable reagents.

The concept of DNA sequencing-by-synthesis (SBS) was revealed in 1988 with an attempt to sequence DNA by detecting the pyrophosphate group that is generated when a nucleotide is incorporated by a DNA polymerase reaction (Hyman (1999) Anal. Biochem. 174: 423-436). Subsequent SBS technologies were based on additional ways to detect the incorporation of a nucleotide to a growing DNA strand. In general, conventional SBS uses an oligonucleotide primer designed to anneal to a predetermined position of the sample template molecule to be sequenced. The primer-template complex is presented with a nucleotide in the presence of a polymerase enzyme. If the nucleotide is complementary to the position on the sample template molecule that is directly 3′ of the end of the oligonucleotide primer, then the DNA polymerase will extend the primer with the nucleotide. The incorporation of the nucleotide and the identity of the inserted nucleotide can then be detected by, e.g., the emission of light, a change in fluorescence, a change in pH (see, e.g., U.S. Pat. No. 7,932,034), a change in enzyme conformation, or some other physical or chemical change in the reaction (see, e.g., WO 1993/023564 and WO 1989/009283; Seo et al. (2005) “Four-color DNA sequencing by synthesis on a chip using photocleavable fluorescent nucleotides,” PNAS 102: 5926-59). Upon each successful incorporation of a nucleotide, a signal is detected that reflects the occurrence, identity, and number of nucleotide incorporations. Unincorporated nucleotides can then be removed (e.g., by chemical degradation or by washing) and the next position in the primer-template can be queried with another nucleotide species.

It is a goal to generate high quality data at a reasonable cost and deliver next-generation sequencing data accurately and rapidly in an easy to use system. Companies such as PacBio have developed specific chemistries for implementation on their systems. At the same time, other companies such as VisiGen and Life Technologies have pursued alternative chemistries for addressing low cost sequencing.

In particular, LaserGen has developed approaches using optical detection systems and certain reaction chemistries to produce and polymerize photo-deprotectable nucleotides that could be employed in next generation sequencing applications, e.g., as described in U.S. Pat. Nos. 7,893,227; 7,897,737; 7,964,352; and 8,148,503. The LaserGen nucleotides have a photocleavable, fluorescent terminator moiety attached to the nucleotide base and a non-blocked 3′ hydroxyl on the ribose sugar. The photocleavable, fluorescent terminator provides a substrate for polymerization, e.g., a polymerase adds the nucleotide analog to the 3′ hydoxyl of the synthesized strand. While attached to the nucleotide at the 3′ end, the photocleavable, fluorescent terminator prevents additional nucleotide addition by the polymerase. Also, the fluorescent moiety provides for identification of the nucleotide added using an excitation light source and a fluorescence emission detector. Upon exposure to a light source of the appropriate wavelength, the light cleaves the photocleavable, fluorescent terminator from the 3′ end of the strand, thus removing the block to synthesis and another nucleotide analog is added to begin the cycle again. When used in a sequencing-by-synthesis reaction, the LaserGen fluorescently labeled nucleotide compounds offer a way to photodeprotect and at the same time allow for extension, e.g., by sterically unblocking the region in the enzyme so as to permit extension.

While these technologies have advanced the field of sequencing, additional systems and methods are needed to improve efficiency, cost, ease-of-use, informativeness, and breadth of application.

### SUMMARY

Provided herein are new classes of photocleavable deoxynucleotides that allow for more precise control over deprotection kinetics compared to previously described compounds. The compounds further feature more favorable solubility properties. The nucleotides find use in methods such as next-generation sequencing. A series of molecules are provided with defined organic substituents that allow fine tuning of the deprotection kinetics when irradiated with an appropriate light source.

For example, in some embodiments, provided herein are compounds comprising the structure:

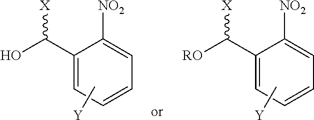

wherein Y is alkoxy (except methoxy), aryloxy, cycloalkyl, cycloalkenyl, amido, alkyl amime, aryl amine, primary alkyl alcohol, primary alkenyl alcohol, secondary alkyl alcohol, secondary alkenyl alcohol, alkyl siloxane, alkenyl siloxane, alkyl silane, and alkenyl silane; R is an organic group, and X is a bulky group. In some embodiments, Y is —OCH₃, —OC₂H₅, —O(CH₂)₂CH₃, —O(CH₂)₃CH₃, —O(CH₂)₄CH₃, —OCH₂CHCH₂, —OC₆H₅, -cycloproply, -cyclobuyl, -cyclopentyl, —NHCONH₂, —N(C₆H₅)₂, —CH₂CH(OH)CH₃, —OSi(CH₃)₃, or —CH₂Si(CH₃)₃. In some embodiments, X is a branched alkyl or a cycloalkyl group. In some embodiments, R comprises a nucleotide base (A, T, C, G, U, etc.). In some embodiments, R comprises a sugar. In some embodiments, R comprise a polynucleotide. In some embodiments, R comprises a detectable moeity (e.g., a fluorescent label).

Also provided herein are compositions (e.g., reaction mixtures and kits) comprising any of the compositions. In some embodiments, the kits further provide nucleic acid sequencing reagents.

In some embodiments, sets of the compounds are provided (e.g., in kits) where the sets contain two or more compounds differing in the identity of the Y group. In some embodiments, the differening Y groups have similar Hammett sigma constants (e.g., differing by 0.3 or less, 0.2 or less, 0.1 or less, etc.).

Further provided herein are methods employing the compounds individually or in sets. In some embodiments, the methods comprise the step of adding a compound to a nucleic acid molecule (e.g., an extended primer in a sequencing reaction). In some embodiments, after additions, the method comprises the step of irradiating the added compound with a light source (e.g., to deprotect the compound).

### DETAILED DESCRIPTION

Provided herein are new classes of photocleavable deoxynucleotides that allow for more precise control over deprotection kinetics compared to previously described compounds. The compounds further feature more favorable solubility properties. The nucleotides find use in methods such as next-generation sequencing. A series of molecules are provided with defined organic substituents that allow fine tuning of the deprotection kinetics when irradiated with an appropriate light source.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, a “nucleotide” comprises a “base” (alternatively, a “nucleobase” or “nitrogenous base”), a “sugar” (in particular, a five-carbon sugar, e.g., ribose or 2-deoxyribose), and a “phosphate moiety” of one or more phosphate groups (e.g., a monophosphate, a diphosphate, or a triphosphate consisting of one, two, or three linked phosphates, respectively). Without the phosphate moiety, the nucleobase and the sugar compose a “nucleoside”. A nucleotide can thus also be called a nucleoside monophosphate or a nucleoside diphosphate or a nucleoside triphosphate, depending on the number of phosphate groups attached. The phosphate moiety is usually attached to the 5-carbon of the sugar, though some nucleotides comprise phosphate moieties attached to the 2-carbon or the 3-carbon of the sugar. Nucleotides contain either a purine (in the nucleotides adenine and guanine) or a pyrimidine base (in the nucleotides cytosine, thymine, and uracil). Ribonucleotides are nucleotides in which the sugar is ribose. Deoxyribonucleotides are nucleotides in which the sugar is deoxyribose.

As used herein, a “nucleic acid” shall mean any nucleic acid molecule, including, without limitation, DNA, RNA, and hybrids thereof. The nucleic acid bases that form nucleic acid molecules can be the bases A, C, G, T and U, as well as derivatives thereof. Derivatives of these bases are well known in the art. The term should be understood to include, as equivalents, analogs of either DNA or RNA made from nucleotide analogs. The term as used herein also encompasses cDNA, that is complementary, or copy, DNA produced from an RNA template, for example by the action of a reverse transcriptase. It is well known that DNA (deoxyribonucleic acid) is a chain of nucleotides comprising 4 types of nucleotides—A (adenine), T (thymine), C (cytosine), and G (guanine)—and that RNA (ribonucleic acid) is a chain of nucleotides consisting of 4 types of nucleotides—A, U (uracil), G, and C. It is also known that all of these 5 types of nucleotides specifically bind to one another in combinations called complementary base pairing. That is, adenine (A) pairs with thymine (T) (in the case of RNA, however, adenine (A) pairs with uracil (U)), and cytosine (C) pairs with guanine (G), so that each of these base pairs forms a double strand. As used herein, “nucleic acid sequencing data”, “nucleic acid sequencing information”, “nucleic acid sequence”, “genomic sequence”, “genetic sequence”, “fragment sequence”, or “nucleic acid sequencing read” denotes any information or data that is indicative of the order of the nucleotide bases (e.g., adenine, guanine, cytosine, and thymine/uracil) in a molecule (e.g., a whole genome, a whole transcriptome, an exome, oligonucleotide, polynucleotide, fragment, etc.) of DNA or RNA

Reference to a base, a nucleotide, or to another molecule may be in the singular or plural. That is, “a base” may refer to a single molecule of that base or to a plurality of the base, e.g., in a solution.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

As used herein, the phrase “dNTP” means deoxynucleotidetriphosphate, where the nucleotide comprises a nucleotide base, such as A, T, C, G or U.

The term “monomer” as used herein means any compound that can be incorporated into a growing molecular chain by a given polymerase. Such monomers include, without limitations, naturally occurring nucleotides (e.g., ATP, GTP, TTP, UTP, CTP, dATP, dGTP, dTTP, dUTP, dCTP, synthetic analogs), precursors for each nucleotide, non-naturally occurring nucleotides and their precursors or any other molecule that can be incorporated into a growing polymer chain by a given polymerase.

As used herein, “complementary” generally refers to specific nucleotide duplexing to form canonical Watson-Crick base pairs, as is understood by those skilled in the art.

However, complementary also includes base-pairing of nucleotide analogs that are capable of universal base-pairing with A, T, G or C nucleotides and locked nucleic acids that enhance the thermal stability of duplexes. One skilled in the art will recognize that hybridization stringency is a determinant in the degree of match or mismatch in the duplex formed by hybridization.

As used herein, “moiety” refers to one of two or more parts into which something may be divided, such as, for example, the various parts of a tether, a molecule or a probe.

A “polymerase” is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase 1, Klenow fragment, Thermophilus aquaticus DNA polymerase, Tth DNA polymerase, Vent DNA polymerase (New England Biolabs), Deep Vent DNA polymerase (New England Biolabs), Bst DNA Polymerase Large Fragment, Stoeffel Fragment, 9° N DNA Polymerase, Pfu DNA Polymerase, Tfl DNA Polymerase, RepliPHI Phi29 Polymerase, Tli DNA polymerase, eukaryotic DNA polymerase beta, telomerase, Therminator polymerase (New England Biolabs), KOD HiFi. DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in U.S. Pat. Appl. Pub. No. 2007/0048748 and in U.S. Pat. Nos. 6,329,178; 6,602,695; and 6,395,524. These polymerases include wild-type, mutant isoforms, and genetically engineered variants such as exo-polymerases and other mutants, e.g., that tolerate labeled nucleotides and incorporate them into a strand of nucleic acid.

The term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the terms “alkyl” and the prefix “alk-” are inclusive of both straight chain and branched chain saturated or unsaturated groups, and of cyclic groups, e.g., cycloalkyl and cycloalkenyl groups. Unless otherwise specified, acyclic alkyl groups are from 1 to 6 carbons. Cyclic groups can be monocyclic or polycyclic and preferably have from 3 to 8 ring carbon atoms. Exemplary cyclic groups include cyclopropyl, cyclopentyl, cyclohexyl, and adamantyl groups. Alkyl groups may be substituted with one or more substituents or unsubstituted. Exemplary substituents include alkoxy, aryloxy, sulfhydryl, alkylthio, arylthio, halogen, alkylsilyl, hydroxyl, fluoroalkyl, perfluoralkyl, amino, aminoalkyl, disubstituted amino, quaternary amino, hydroxyalkyl, carboxyalkyl, and carboxyl groups. When the prefix “alk” is used, the number of carbons contained in the alkyl chain is given by the range that directly precedes this term, with the number of carbons contained in the remainder of the group that includes this prefix defined elsewhere herein. For example, the term “C₁-C₄ alkaryl” exemplifies an aryl group of from 6 to 18 carbons (e.g., see below) attached to an alkyl group of from 1 to 4 carbons.

As used herein, the term “aryl” refers to a carbocyclic aromatic ring or ring system. Unless otherwise specified, aryl groups are from 6 to 18 carbons. Examples of aryl groups include phenyl, naphthyl, biphenyl, fluorenyl, and indenyl groups.

As used herein, the term “heteroaryl” refers to an aromatic ring or ring system that contains at least one ring heteroatom (e.g., O, S, Se, N, or P). Unless otherwise specified, heteroaryl groups are from 1 to 9 carbons. Heteroaryl groups include furanyl, thienyl, pyrrolyl, imidazolyl, pyrazolyl, oxazolyl, isoxazolyl, thiazolyl, isothiazolyl, triazolyl, tetrazolyl, oxadiazolyl, oxatriazolyl, pyridyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl groups.

As used herein, the term “heterocycle” refers to a non-aromatic ring or ring system that contains at least one ring heteroatom (e.g., O, S, Se, N, or P). Unless otherwise specified, heterocyclic groups are from 2 to 9 carbons. Heterocyclic groups include, for example, dihydropyrrolyl, tetrahydropyrrolyl, piperazinyl, pyranyl, dihydropyranyl, tetrahydropyranyl, dihydrofuranyl, tetrahydrofuranyl, dihydrothiophene, tetrahydrothiophene, and morpholinyl groups.

Aryl, heteroaryl, or heterocyclic groups may be unsubstituted or substituted by one or more substituents selected from the group consisting of C₁₋₆ alkyl, hydroxy, halo, nitro, C₁₋₆ alkoxy, C₁₋₆ alkylthio, trifluoromethyl, C₁₋₆ acyl, arylcarbonyl, heteroarylcarbonyl, nitrile, C₁₋₆ alkoxycarbonyl, alkaryl (where the alkyl group has from 1 to 4 carbon atoms), and alkheteroaryl (where the alkyl group has from 1 to 4 carbon atoms).

As used herein, the term “alkoxy” refers to a chemical substituent of the formula —OR, where R is an alkyl group. By “aryloxy” is meant a chemical substituent of the formula —OR′, where R′ is an aryl group.

As used herein, a “bulky group” refers to a chemical group that provides steric hindrance, including, but not limited to, branched alkyl groups having three or more carbons (e.g., i-propyl, i-butyl, t-butyl, i-pentyl, t-pentyl, i-hexyl or t-hexyl group), substituted or unsubstituted cyclic C5-6 alkyl groups (e.g. cyclopentane, cyclohexane, cyclopentene, cyclohexene, 1,2-cyclohexadiene, 1,3-cyclohexadiene or 1,4-cyclohexadiene), and substituted or unsubstituted aryl groups (e.g., phenyl, benzyl, tolyl or xylyl groups).

As used herein, a “system” denotes a set of components, real or abstract, comprising a whole where each component interacts with or is related to at least one other component within the whole.

### Embodiments

In some embodiments, provided herein is a new chemical class of photo-deprotectable nucleotide compounds that contain specific functional groups located on a 2-nitrophenyl group that have similar electron donating properties, as described by the Hammett sigma constants. In some embodiments, provided herein are a series of photodeprotectable groups for use in nucleic acid assays such as nucleic acid sequencing comprising or consisting of one of the following structures:

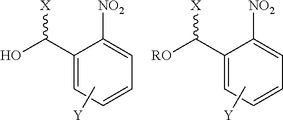

Where R=any organic group including, but not limited to, deoxynucleotide triphosphates, X=a bulky group attached to the benzyl carbon where the group is present as a racemate or in a chiral R- or S-enantiomcric configuration, and Y=one of a series of related functional groups with closely spaced Hammett σ-para values. The Y group may alternatively be at the 3-, 4-, 5- or 6-position of the phenyl ring.

A series of photocleavable deoxynucleotides has recently been described by Metzker, et al. known as Lightning Terminators (see e.g., Stupi et al., Angew. Chem. Int. Ed., (51), 1-5 (2012); U.S. Pat. No. 7,897,737, herein incorporated by reference in its entirety). These compounds were designed for next-generation sequencing purposes using Sequencing-by-Synthesis (SBS). In SBS, nucleotides are added one at a time in sequential order, followed by base interrogation/detection. These compounds are shown below:

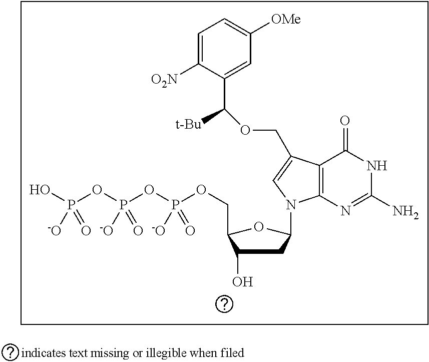

7-[(S)-1-(5-methoxy-2-nitrophenyl)-2,2-dimethylpropyloxy]methyl-7-deaza-2′-deoxyguanosine-5′-triphosphate

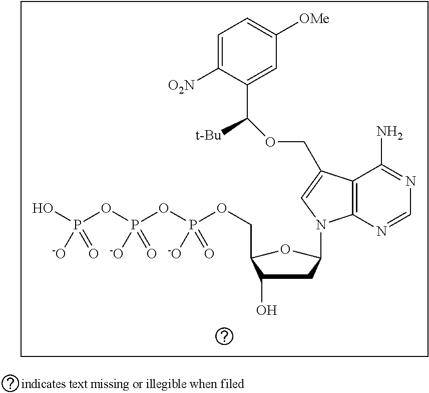

7-[(S)-1-(5-methoxy-2-nitrophenyl)-2,2-dimethylpropyloxy]methyl-7-deaza-2′-deoxyadenosine-5′-triphosphate

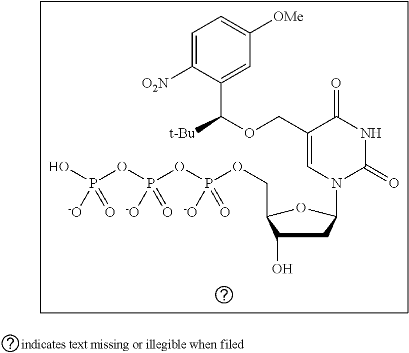

7-[(S)-1-(5-methoxy-2-nitrophenyl)-2,2-dimethylpropyloxy]methyl-7-deaza-2′-deoxyuridine-5′-triphosphate

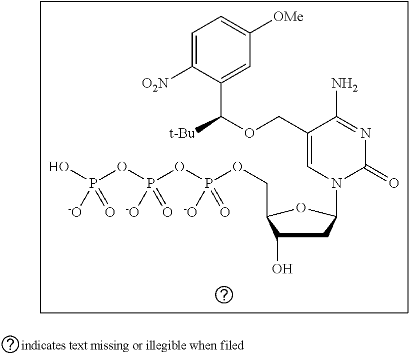

7-[(S)-1-(5-methoxy-2-nitrophenyl)-2,2-dimethylpropyloxy]methyl-7-deaza-2′-deoxycytidine-5′-triphosphate.

These nucleotide analogs can be photodeprotected using a wavelength of approximately 350 nm, which results in release of the 5-methoxy-2-nitrobenzylketone, thereby leaving the exocyclic hydroxyl derivative of the dNTP base:

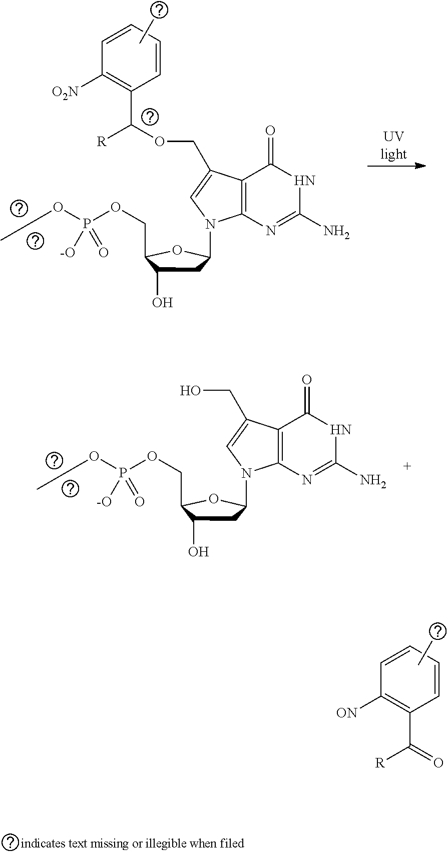

In this example, deprotection of the deoxyguanosine analog is shown. This chemistry is similar for all the nucleotide base analogs. The mechanism for photocleavage of the 2-nitrobenzyl group is:

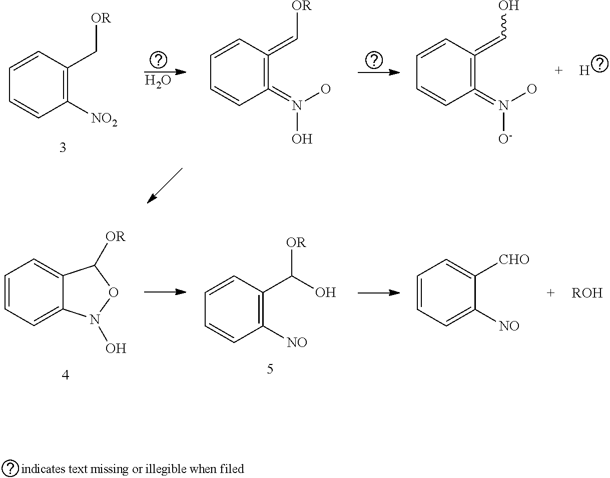

Cleavage proceeds irreversibly from the nitronic acid complex, which forms via excitation of the nitro group. After cyclization to the benzisoxazoline intermediate, formation of the hemiacetal results in cleavage of the nitroso arylaldehyde from the alkyl alcohol. In this example, the R group represents the dNTP analogs.

Metzger, et al. (see e.g., U.S. Pat. No. 7,897,737) has shown that the presence of the 5-methoxy group coupled with the bulky R group in the S-configuration on the benzylic carbon show favorable kinetic deprotection characteristics compared to previous analogs without the methoxy group—i.e. fast deprotection times (<1 sec.). The methoxy group, being electron donating, must cause destabilization of the neutral nitronic acid intermediate, thereby increasing the rate of cleavage.

In U.S. Pat. No. 7,897,737, dNTP analogs are described containing a variety of functional groups on the 2-nitrophenyl ring, including —OMe, —OH, —NO₂, —CN, halides, straight chain and branched alkyl groups, among others. These groups display a wide variation in electron donating and electron withdrawing properties. For example, the —OH group has a Hammett σ-para value of −0.32, indicating relatively strong electron donating properties (ring activation). In contrast, —CN and —NO₂ groups have Hammett values of +0.66 and +0.778, respectively, indicating relatively strong electron withdrawing properties (ring deactivation). These large differences in Hammett values make it difficult to predict the effect on cleavage kinetics, especially when the 5-methoxy group was found to have optimal deprotection kinetic properties.

Given the superior performance and solubility of the methoxy group, provided herein are a series of alternative dNTP analogs where the cleavage kinetics and solubility properties are fine-tuned to any desirable specification. Stupi (supra.) describes a group of photocleavable dNTP analogs containing the 5-methoxy-2-nitrobenzyl group; these analogs have DT₅₀ (50% deprotection times) of approximately 0.7 seconds. These molecules do not provide flexibility for slightly faster or slightly slower deprotection kinetics so as to allow researchers to adjust the deprotection kinetics in a logical fashion. The systems and methods herein provide such capability and flexibility.

The present invention solves the challenge of providing such compounds, which concomitantly have desirable solubility properties by substituting the methoxy group with alternative groups having similar ring activating and solubility properties. This is accomplished by selecting functional groups with similar Hammett σ-para values.

A partial list of suitable functional groups is provided in Table 1, below.

TABLE 1

| Functional Group | Hammett o-para |
|---|---|
| —OCH₃ | −0.27 |
| —OC₂H₃ | −0.24 |
| —O(CH₂)₂CH₃ | −0.25 |
| —O(CH₂)₃CH₃ | −0.32 |
| —O(CH₂)₃CH₃ | −0.34 |
| —OCH₂CHCH₂ | −0.25 |
| —OC₆H₅ | −0.32 |
| -cyclopropyl | −0.21 |
| -cyclobutyl | −0.14 |
| -cyclopenyl | −0.14 |
| —NHCONH₂ | −0.24 |
| —N(C₆H₃)₂ | −0.22 |
| —CH₂CH(OH)CH₃ | −0.17 |
| —OSi(CH₃)₃ | −0.27 |
| —CH₂Si(CH₃)₂ | −0.27 |

Provided herein are a series of compounds comprising ring substituents belonging to the groups listed above to allow for high-resolution fine tuning of deprotection kinetics for dNTP analogs containing the 2-nitrobenzyl group attached to any nucleotide base. Specifically, provided herein are compounds comprising any group belonging to the following general functional categories: alkoxy (except methoxy), aryloxy, cycloalkyl, cycloalkenyl, amido, alkyl amime, aryl amine, primary alkyl alcohol, primary alkenyl alcohol, secondary alkyl alcohol, secondary alkenyl alcohol, alkyl siloxane, alkenyl siloxane, alkyl silane, and alkenyl silane. The position of the above-described groups may be on the 3-, 4-, 5- or 6-position of the phenyl ring system. A label (e.g., optical or electrochemical label) may be also attached to the nucleotide analogs.

An example showing the deoxyuridine derivative is illustrated below. In this case, the R group denotes any of the above mentioned organic groups. The same photodeprotection group may be linked to any of the naturally occurring nucleotide bases. The t-butyl group located on the benzylic carbon can also be substituted with other bulky groups including, but not limited to, cycloalkyl groups.

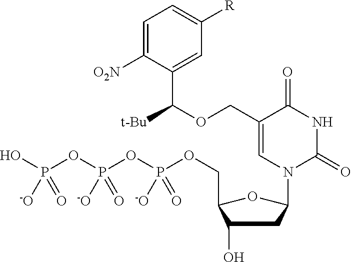

Provided herein are nucleic acid molecules incorporating the nucleic analogs herein (e.g., extended sequencing primers).

Also provided herein are compositions (e.g., reaction mixtures) and kits comprising one or more of the nucleotide analogs described herein. Kits may comprise sets (e.g., 2 or more, 3 or more, 4 or more, 5 or more, etc.) of different nucleotide analogs to allow the user to finely tune reactions (e.g., multiplex reactions) to the desired parameters. Kit may further comprise buffers, enzymes (e.g., polymerases), labels, or other reagents useful, sufficient, or necessary for carrying out a nucleic acid analysis technique (e.g., amplification, sequencing, etc.). Kits may further comprise appropriate positive and negative control reagents, instructions, containers, instruments, and software (e.g., for analyzing and reported data generated from an assay) for the desired assay or reaction. Kits may be used for research or clinical (e.g., diagnostic) indications.

The described nucleotide analogs may be used in a variety of different applications. Some examples include nucleic acid labeling and next-generation sequencing, including Sequencing-by Synthesis (SBS), Sequencing-by-Ligation (SBL), real-time sequencing using either Total Internal Reflection Microscopy or zero-mode waveguide detection. These analogs may be used for their polymerization terminating properties, as with Lasergen's Lightning Terminators, however, the described R phenyl groups provided herein allow one to adjust and control deprotection kinetics and enzyme selectivity to a greater extent than the nucleotide analogs previously available.

In some embodiments, the nucleotide analogs described herein are used to perform SBS sequencing coupled with zeromode waveguide detection where there is no need to wash the flow cell in between base additions. In this mode, all four fluorescently-labeled nucleotide analogs are added to a sequencing cell containing multiple zero-mode waveguide (ZMW) cells. An optical detector is used to monitor incorporation of any base into the growing nucleotide chain, since these nucleotide analogs have self-terminating properties and, therefore, terminate after incorporation. After detection, highly localized deprotection in ZMW cells with an appropriate light source allow for the next base to be incorporated, followed by another round of detection. The presence of a ZMW disposable and evanescent optical waveguide allows for only a very small volume of tile total reaction volume to be illuminated at any one time, thus most of nucleotides in solution remain labeled.

In this and many other sequencing formats, deprotection times and enzyme selectivity play an important role in determining sequencing efficiency and accuracy. Rapid deprotection times and high enzyme selectivity are desirable attributes for next-generation sequencing. The compounds described herein are an improvement over previous compounds in that they allow one to very accurately adjust the chemical properties of the labeled nucleotide analogs to meet required specifications for deprotection times and enzyme selectivity. By using functional groups that display closely-related electron-donating ring activation properties, this process becomes much easier than substituting with different functional groups that display widely varying electron withdrawing or donating properties.

### Zero Mode Wave Guides

In some assays, molecules are confined in a series, array, or other arrangement of small holes, pores, or wells, for example, a zero mode waveguide (ZMW), e.g., as described in U.S. Pat. Appl. Pub. No. 2011/0117637, incorporated herein by reference. ZMW arrays have been applied to a range of biochemical analyses and have found particular usefulness for genetic analysis. ZMWs typically comprise a nanoscale core, well, or opening disposed in an opaque cladding layer that is disposed upon a transparent substrate, e.g., a circular hole in an aluminum cladding film deposited on a clear silica substrate. See, e.g., J. Korlach et al, “Selective aluminum passivation for targeted immobilization of single DNA polymerase molecules in zero-mode waveguide nanostructures”, 105 PNAS 1176-81 (2008). A typical ZMW hole is ˜70 nm in diameter and ˜100 nm in depth. ZMW technology allows the sensitive analysis of single molecules because, as light travels through a small aperture, the optical field decays exponentially inside the chamber. That is, due to the narrow dimensions of the well, electromagnetic radiation that is of a frequency above a particular cut-off frequency will be prevented from propagating all the way through the core. Notwithstanding the foregoing, the radiation will penetrate a limited distance into the core, providing a very small illuminated volume within the core. By illuminating a very small volume, one can potentially interrogate very small quantities of reagents, including, e.g., single molecule reactions. The observation volume within an illuminated ZMW is ˜20 zeptoliters (20×10-21 liters). Within this volume, the activity of DNA polymerase incorporating a single nucleotide can be readily detected.

By monitoring reactions at the single molecule level, one can precisely identify and/or monitor a given reaction. In particular, the technology is the basis for a particularly promising field of single molecule DNA sequencing technology that monitors the molecule-by-molecule (e.g., nucleotide-by-nucleotide) synthesis of a DNA strand in a template-dependent fashion by a single polymerase enzyme (e.g., Single Molecule Real Time (SMRT) DNA Sequencing as performed, e.g., by a Pacific Biosciences RS Sequencer (Pacific Biosciences, Menlo Park, Calif.)). See, e.g., U.S. Pat. Nos. 7,476,503; 7,486,865; 7,907,800; and 7,170,050; and U.S. patent application Ser. Nos. 12/553,478, 12/767,673; 12/814,075; 12/413,258; and 12/413,466, each incorporated herein by reference in its entirety for all purposes. See also, Eid, J. et al. 2009. “Real-time DNA sequencing from single polymerase molecules”, 323 Science: 133-38 (2009); Korlach, J. et al. “Long, processive enzymatic DNA synthesis using 100% dye-labeled terminal phosphate-linked nucleotides”, 27 Nucleosides, Nucleotides & Nucleic Acids: 1072-82 (2008); Lundquist, P. M. et al., “Parallel confocal detection of single molecules in real time”, 33 Optics Letters: 1026-28 (2008); Korlach, J. et al., “Selective aluminum passivation for targeted immobilization of single dna polymerase molecules in zero-mode waveguide nanostructures”, 105 Proc Natl Acad Sci USA: 1176-81 (2008); Foquet, M. et al., “Improved fabrication of zero-mode waveguides for single-molecule detection”, 103 Journal of Applied Physics (2008); and Levene, M. J. et al. “Zero-mode waveguides for single-molecule analysis at high concentrations”, 299 Science: 682-86 (2003), each incorporated herein by reference in its entirety for all purposes.

### Sequencing Methods

The technology relates, in some embodiments, to methods for sequencing a nucleic acid. In some embodiments, sequencing is performed by the following sequence of events.

First, a nucleotide analog is added to the 3′ end of a growing strand by the polymerase, e.g., by the enzyme-catalyzed attack of the 3′ hydroxyl on the alpha-phosphate of the nucleotide analog. Further extension of the strand by the polymerase is blocked by the 3′ terminating group on the incorporated nucleotide analog. A detectable moiety on the incorporated nucleotide is queried or the incorporated nucleotide is otherwise detected.

Then, the terminating moiety is removed by exposure (e.g., in the illumination volume of a zero mode waveguide) to a wavelength of light that cleaves the terminating moiety from the nucleotide analog. The 3′ hydroxyl of the growing strand is free for further polymerization: the next base is incorporated to continue another cycle, e.g., a nucleotide analog is oriented in the polymerase active site, the nucleotide analog is added to the 3′ end of the growing strand by the polymerase, the nucleotide analog is queried to identify the base added, and the nucleotide analog is deprotected.

In some embodiments of the technology, nucleic acid sequence data are generated. Various embodiments of nucleic acid sequencing platforms (e.g., a nucleic acid sequencer) include components as described below. According to various embodiments, a sequencing instrument includes a fluidic delivery and control unit, a sample processing unit, a signal detection unit, and a data acquisition, analysis and control unit. Various embodiments of the instrument provide for automated sequencing that is used to gather sequence information from a plurality of sequences in parallel and/or substantially simultaneously.

In some embodiments, the fluidics delivery and control unit includes a reagent delivery system. The reagent delivery system includes a reagent reservoir for the storage of various reagents. The reagents can include RNA-based primers, forward/reverse DNA primers, nucleotide mixtures (e.g., compositions comprising nucleotide analogs as provided herein) for sequencing-by-synthesis, buffers, wash reagents, blocking reagents, stripping reagents, and the like. Additionally, the reagent delivery system can include a pipetting system or a continuous flow system that connects the sample processing unit with the reagent reservoir.

In some embodiments, the sample processing unit includes a sample chamber, such as flow cell, a substrate, a micro-array, a multi-well tray, or the like. The sample processing unit can include multiple lanes, multiple channels, multiple wells, or other means of processing multiple sample sets substantially simultaneously. Additionally, the sample processing unit can include multiple sample chambers to enable processing of multiple runs simultaneously.

In particular embodiments, the system can perform signal detection on one sample chamber while substantially simultaneously processing another sample chamber. Additionally, the sample processing unit can include an automation system for moving or manipulating the sample chamber. In some embodiments, the signal detection unit can include an imaging or detection sensor. For example, the imaging or detection sensor can include a CCD, a CMOS, an ion sensor, such as an ion sensitive layer overlying a CMOS, a current detector, or the like. The signal detection unit can include an excitation system to cause a probe, such as a fluorescent dye, to emit a signal. The detection system can include an illumination source, such as arc lamp, a laser, a light emitting diode (LED), or the like. In particular embodiments, the signal detection unit includes optics for the transmission of light from an illumination source to the sample or from the sample to the imaging or detection sensor.

It will be appreciated by one skilled in the art that various embodiments of the instruments and systems are used to practice sequencing methods such as sequencing by synthesis, single molecule methods, and other sequencing techniques.

In some embodiments, the sequencing instrument determines the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In some embodiments, the nucleic acid can include or be derived from a fragment library, a mate pair library, a ChIP fragment, or the like. In particular embodiments, the sequencing instrument can obtain the sequence information from a single nucleic acid molecule or from a group of substantially identical nucleic acid molecules.

In some embodiments, the sequencing instrument can output nucleic acid sequencing read data in a variety of different output data file types/formats, including, but not limited to: *.txt, *.fasta, *.csfasta, *seq.txt, *qseq.txt, *.fastq, *.sff, *prb.txt, *.sms, *srs, and/or *.qv.

Some embodiments provide a system for reconstructing a nucleic acid sequence. The system can include a nucleic acid sequencer, a sample sequence data storage, a reference sequence data storage, and an analytics computing device/server/node. In some embodiments, the analytics computing device/server/node can be a workstation, mainframe computer, personal computer, mobile device, etc. The nucleic acid sequencer can be configured to analyze (e.g., interrogate) a nucleic acid fragment (e.g., single fragment, mate-pair fragment, paired-end fragment, etc.) utilizing all available varieties of techniques, platforms or technologies to obtain nucleic acid sequence information, in particular the methods as described herein using compositions provided herein. In some embodiments, the nucleic acid sequencer is in communications with the sample sequence data storage either directly via a data cable (e.g., serial cable, direct cable connection, etc.) or bus linkage or, alternatively, through a network connection (e.g., Internet, LAN, WAN, VPN, etc.).

In some embodiments, the sample sequence data storage is any database storage device, system, or implementation (e.g., data storage partition, etc.) that is configured to organize and store nucleic acid sequence read data generated by nucleic acid sequencer such that the data can be searched and retrieved manually (e.g., by a database administrator or client operator) or automatically by way of a computer program, application, or software script. In some embodiments, the reference data storage can be any database device, storage system, or implementation (e.g., data storage partition, etc.) that is configured to organize and store reference sequences (e.g., whole or partial genome, whole or partial exome, SNP, gen, etc.) such that the data can be searched and retrieved manually (e.g., by a database administrator or client operator) or automatically by way of a computer program, application, and/or software script. In some embodiments, the sample nucleic acid sequencing read data can be stored on the sample sequence data storage and/or the reference data storage in a variety of different data file types/formats, including, but not limited to: *.txt, *.fasta, *.csfasta, *seq.txt, *qseq.txt, *.fastq, *.sff, *prb.txt, *.sms, *srs and/or *.qv.

In some embodiments, the sample sequence data storage and the reference data storage are independent standalone devices/systems or implemented on different devices. In some embodiments, the sample sequence data storage and the reference data storage are implemented on the same device/system. In some embodiments, the sample sequence data storage and/or the reference data storage can be implemented on the analytics computing device/server/node. The analytics computing device/server/node can be in communications with the sample sequence data storage and the reference data storage either directly via a data cable (e.g., serial cable, direct cable connection, etc.) or bus linkage or, alternatively, through a network connection (e.g., Internet, LAN, WAN, VPN, etc.). In some embodiments, analytics computing device/server/node can host a reference mapping engine, a de novo mapping module, and/or a tertiary analysis engine. In some embodiments, the reference mapping engine can be configured to obtain sample nucleic acid sequence reads from the sample data storage and map them against one or more reference sequences obtained from the reference data storage to assemble the reads into a sequence that is similar but not necessarily identical to the reference sequence using all varieties of reference mapping/alignment techniques and methods. The reassembled sequence can then be further analyzed by one or more optional tertiary analysis engines to identify differences in the genetic makeup (genotype), gene expression or epigenetic status of individuals that can result in large differences in physical characteristics (phenotype). For example, in some embodiments, the tertiary analysis engine can be configured to identify various genomic variants (in the assembled sequence) due to mutations, recombination/crossover or genetic drift. Examples of types of genomic variants include, but are not limited to: single nucleotide polymorphisms (SNPs), copy number variations (CNVs), insertions/deletions (Indels), inversions, etc. The optional de novo mapping module can be configured to assemble sample nucleic acid sequence reads from the sample data storage into new and previously unknown sequences. It should be understood, however, that the various engines and modules hosted on the analytics computing device/server/node can be combined or collapsed into a single engine or module, depending on the requirements of the particular application or system architecture. Moreover, in some embodiments, the analytics computing device/server/node can host additional engines or modules as needed by the particular application or system architecture.

Although the disclosure herein refers to certain illustrated embodiments, it is to be understood that these embodiments are presented by way of example and not by way of limitation.

### Example

### Synthesis of Photocleavable 2-nitrobenzyl-5-ethoxy Analog of Deoxyuridine Triphosphate

The following example shows how to synthesize the compound shown above where R=ethoxy. Similar strategies are followed for synthesizing other pyrimidine base analogs, including deoxycytidine. The overall strategy is also described in reference (Stupi et al.) for the methoxy compound. For the ethoxy compound, the synthesis can be started from commercially available 3-iodo-4-nitrophenol.

### Preparation of starting material 3-iodo-4.nitrophenetole

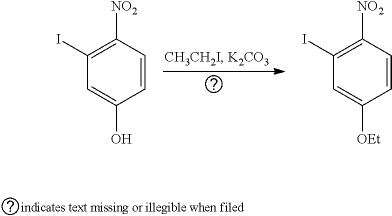 [SMILES: O=[N+]([O-])c1ccc(O)cc1I ; CCOc1ccc([N+](=O)[O-])c(I)c1]

### Conversion of starting material to racemic 1-(5-ethoxy-2-nitrophenyl)-2,2-dimethyl-1propanol

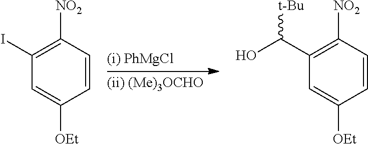 [SMILES: CCOc1ccc([N+](=O)[O-])c(I)c1 ; CCOc1ccc([N+](=O)[O-])c(C(O)C(C)(C)C)c1]

### Fractional Crystallization of S-Camphanate Ester

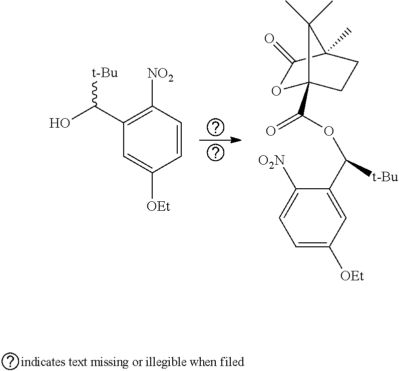 [SMILES: CCOc1ccc([N+](=O)[O-])c(C(O)C(C)(C)C)c1 ; CCOc1ccc([N+](=O)[O-])c([C@@H](OC(=O)[C@@]23CC[C@@](C)(C(=O)O2)C3(C)C)C(C)(C)C)c1]

### Hydrolysis of S-camphanate ester to enantiopure (S)-1-(5-ethoxy-2-nitrophenyl)-2,2dimethyl-1-propanol

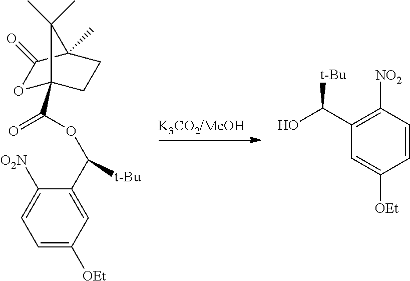 [SMILES: CCOc1ccc([N+](=O)[O-])c([C@@H](OC(=O)[C@@]23CC[C@@](C)(C(=O)O2)C3(C)C)C(C)(C)C)c1 ; CCOc1ccc([N+](=O)[O-])c([C@@H](O)C(C)(C)C)c1]

### Coupling of (S)-1-(5-ethoxy-2-nitrophenyl)-2,2-dimethyl-1-propanol to 5-bromomethyl deoxyuridine intermediate

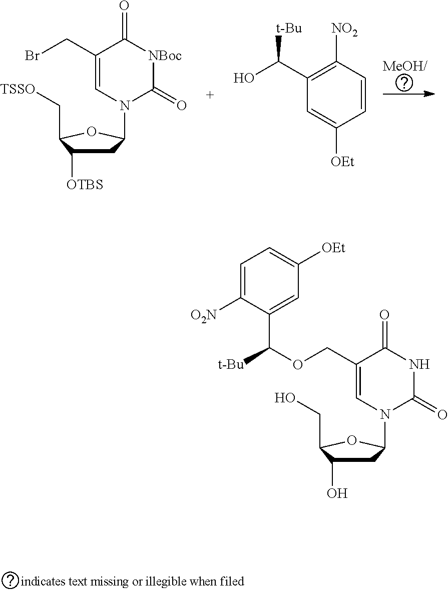

### Synthesis of 5-[(S)-1-(5-ethoxy-2-nitrophenyl)-2,2-dimethylpropyloxy]methyl-2′deoxyuridine-S′-triphosphate

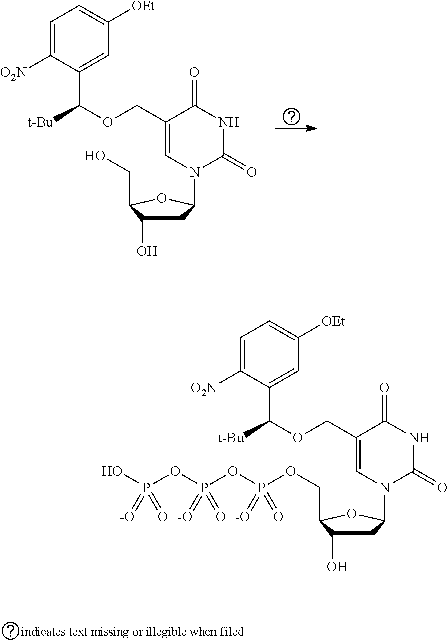

In this example, t-butyl was used as the bulky stcric group on the benzylic carbon. This group may be substituted with other groups, depending on the properties needed or desired for enzymatic activity, kinetics and selectivity. Similar synthetic routes may be utilized for the synthesis of other pyrimidine-based nucleotides, such as deoxycytidine.

1-18. (canceled)

19. A compound comprising the structure: 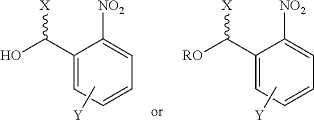
wherein Y is selected from the group consisting of alkoxy comprising 2 or more carbons, aryloxy, cycloalkyl, cycloalkenyl, amido, alkyl amime, aryl amine, primary alkyl alcohol, primary alkenyl alcohol, secondary alkyl alcohol, secondary alkenyl alcohol, alkyl siloxane, alkenyl siloxane, alkyl silane, and alkenyl silane; R is an organic group, and X is a bulky group.

20. The compound of claim 19, wherein Y is selected from the group consisting of —OCH₃, —OC₂H₅, —O(CH₂)₂CH₃, —O(CH₂)₃CH₃, —O(CH₂)₄CH₃, —OCH₂CHCH₂, —OC₆H₅, -cycloproply, -cyclobuyl, -cyclopentyl, —NHCONH₂, —N(C₆H₅)₂, —CH₂CH(OH)CH₃, —OSi(CH₃)₃, and —CH₂Si(CH₃)₃.

21. The compound of claim 19, wherein X is a branched alkyl or cycloalkyl group.

22. The compound of claim 19, wherein R comprises a nucleotide base.

23. The compound of claim 19, wherein R comprises a sugar.

24. The compound of claim 19, wherein R comprise a polynucleotide.

25. The compound of claim 19, wherein R comprises a detectable moeity.

26. The compound of claim 25, wherein said detectable moeity comprises a fluorescent moeity.

27. A composition comprising a compound of claim 19.

28. The composition of claim 27, wherein said compound is in a reaction mixture.

29. The composition of claim 28, further comprising nucleic acid sequencing reagents.

30. A kit comprising a compound of claim 19.

31. The kit of claim 30 comprising a plurality of compounds of claim 19 differering in the identity of the Y group.

32. The kit of claim 31, wherein said differening Y groups differ in Hammett sigma constant by 0.2 or less.

33. A method comprising adding a compound of claim 19 to a nucleic acid molecule.

34. The method of claim 33, further comprising the step of irradiating the added compound with a light source.

35. The method of claim 33 wherein the nucleic acid molecule is an extended sequencing primer and the method further comprises conducting a sequencing reaction.
